(19) United States
(12) Patent Application Publication
(10) Pub. No.: US 2025/0142675 A1
(43) Pub. Date: May 1, 2025

(54) CONSUMABLES HEATED WITHOUT COMBUSTION

(71) Applicant: MARK KRIETZMAN, PALOS VERDES ESTATES, CA (US)

(72) Inventor: MARK KRIETZMAN, PALOS VERDES ESTATES, CA (US)

(21) Appl. No.: 19/007,091

(22) Filed: Dec. 31, 2024

Related U.S. Application Data
(63) Continuation-in-part of application No. 18/927,146, filed on Oct. 25, 2024.
Application No. 18/927,146 is a continuation of application No. 18/592,335, filed on Feb. 29, 2024, now Pat. No. 12,160,935.
Application No. 18/592,335 is a continuation-in-part of application No. 18/133,991, filed on Apr. 12, 2023, now Pat. No. 11,979,949.
Application No. 18/133,991 is a continuation-in-part of application No. 17/211,721, filed on Mar. 24, 2021, now Pat. No. 11,647,566.
Application No. 17/211,721 is a continuation-in-part of application No. 16/118,244, filed on Aug. 30, 2018, now Pat. No. 10,986,872.
Application No. 16/118,244 is a continuation of application No. 15/045,410, filed on Feb. 17, 2016, now Pat. No. 10,076,137.
Application No. 18/133,991 is a continuation-in-part of application No. 17/147,030, filed on Jan. 12, 2021, now Pat. No. 11,770,877.
Application No. 17/147,030 is a continuation of application No. 16/410,858, filed on May 13, 2019, now Pat. No. 10,893,707.
Application No. 16/410,858 is a continuation-in-part of application No. 16/118,244, filed on Aug. 30, 2018, now Pat. No. 10,986,872.
Application No. 16/118,244 is a continuation-in-part of application No. 15/898,629, filed on Feb. 18, 2018, now Pat. No. 10,299,515.
Application No. 15/898,629 is a continuation-in-part of application No. 15/045,442, filed on Feb. 17, 2016, now Pat. No. 9,894,936.
(63) Continuation-in-part of application No. 18/621,058, filed on Mar. 28, 2024.
Application No. 18/621,058 is a continuation of application No. 18/412,194, filed on Jan. 12, 2024, now Pat. No. 12,245,337.
Application No. 18/412,194 is a continuation-in-part of application No. 18/133,991, filed on Apr. 12, 2023, now Pat. No. 11,979,949.
(63) Continuation-in-part of application No. 18/412,194, filed on Jan. 12, 2024, now Pat. No. 12,245,337.
(63) Continuation-in-part of application No. 18/233,240, filed on Aug. 11, 2023.
Application No. 18/233,240 is a continuation-in-part of application No. 17/147,030, filed on Jan. 12, 2021, now Pat. No. 11,770,877.
(60) Provisional application No. 63/621,909, filed on Jan. 17, 2024.
(60) Provisional application No. 63/620,181, filed on Jan. 12, 2024.
(60) Provisional application No. 62/116,926, filed on Feb. 17, 2015.
(60) Provisional application No. 62/127,817, filed on Mar. 3, 2015.
(60) Provisional application No. 62/184,396, filed on Jun. 25, 2015.
(60) Provisional application No. 62/208,786, filed on Aug. 23, 2015.
(60) Provisional application No. 62/270,557, filed on Dec. 21, 2015.
(60) Provisional application No. 62/551,234, filed on Aug. 29, 2017.
(60) Provisional application No. 62/116,926, filed on Feb. 17, 2015.
(60) Provisional application No. 62/127,817, filed on Mar. 3, 2015.
(60) Provisional application No. 62/184,396, filed on Jun. 25, 2015.
(60) Provisional application No. 62/208,786, filed on Aug. 23, 2015.
(60) Provisional application No. 62/270,557, filed on Dec. 21, 2015.
(60) Provisional application No. 63/437,698, filed on Jan. 8, 2023.

(51) Int. Cl. H05B 1/02; A24B 15/16; A24F 40/40; A24F 40/42; A24F 40/46; A24F 40/50; A24F 40/60; H05B 3/14; H05B 3/42

(52) U.S. Cl. CPC H05B 1/0244; A24B 15/16; A24F 40/40; A24F 40/42; A24F 40/46; A24F 40/50; H05B 1/0225; H05B 3/146; H05B 3/42; A24F 40/60; H05B 2203/014; H05B 2203/021; H05B 2203/022

(57) ABSTRACT

A disposable consumable with tobacco, hemp and/or cannabis therein is heated in a receivor allowing conduction and convection. The consumable when inserted into a heating chamber via a receivor and a spacer formed on the consumable separates a portion of the consumable from the receivor wall forming one or more regions of air. In some instances slots or uneven portions of the spacer form air passageways with in the receivor between the consumable and the receivor inner wall.

### RELATED APPLICATIONS

This application claims the benefit of United States (U.S.) Provisional Pat. Application Ser. No. 63/620,181 entitled “VAPORIZING CONSUMABLES HEATED WITH CONVECTION AND CONDUCTION IN A PORTABLE DEVICE” filed Jan. 12, 2024 and U.S. Provisional Pat. Application Ser. No. 63/621,909 entitled “CONSUMABLES HEATED WITH CONVECTION AND CONDUCTION IN A PORTABLE DEVICE” filed Jan. 17, 2024 and the entirety of each application is hereby incorporated by this reference as if fully set forth herein.

This application is a Continuation in Part of U.S. patent application Ser. No. 18/927,146 entitled “HEATING WITHOUT BURNING TOBACCO, IN A DISPOSABLE CONSUMABLE, FOR INHALATION” filed Oct. 25, 2024 and is a Continuation of U.S. Pat. No. 12,160,935 (also Ser. No. 18/592,335) entitled “VAPORIZING CONSUMABLES HEATED WITH CONVECTION AND CONDUCTION IN A PORTABLE DEVICE” filed Feb. 29, 2024. U.S. Pat. No. 12,160,935 is a Continuation In Part of U.S. patent application Ser. No. 18/133,991 (also U.S. Pat. No. 11,979,949) filed Apr. 12, 2023 entitled “PORTABLE MULTIZONE INDUCTION VAPORIZER FOR TOBACCO CONSUMABLES” which is a Continuation of U.S. patent application Ser. No. 17/147,030, (also U.S. Pat. No. 11,707,877) filed Jan. 12, 2021, entitled “PORTABLE TEMPERATURE CONTROLLED AROMATHERAPY VAPORIZERS” which is a Continuation of U.S. Pat. No. 10,893,707, filed May 13, 2019 entitled “Portable Temperature Controlled Aromatherapy Vaporizers” which is a Continuation of U.S. Pat. No. 10,299,515, filed Feb. 18, 2018 entitled “Dynamic Zoned Vaporizer” which claimed the benefit of U.S. Provisional Application Ser. No. 62/551,234 entitled “ZONED VAPORIZERS” filed Aug. 29, 2017 and is a Continuation in part of U.S. Pat. No. 9,894,936, filed Feb. 16, 2016 and entitled “Zoned Vaporizer” U.S. Pat. No. 9,894,936 which claimed the benefit of U.S. Provisional Pat. Applications Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications are incorporated by reference herein in their entirety as if fully set forth herein.

U.S. Pat. No. 10,893,707 is also a Continuation In Part of U.S. Pat. No. 10,986,872 (also Ser. No. 16/188,244) entitled “VAPORIZER AND VAPORIZER CARTRIDGES” filed Aug. 30, 2018 which is a Continuation of U.S. Pat. No. 10,076,137 entitled “VAPORIZER AND VAPORIZER” CARTRIDGES filed Feb. 17, 2016 and which claimed the benefit of U.S. Provisional Pat. Applications Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications and patents are incorporated by reference herein in their entirety as if fully set forth herein.

U.S. patent application Ser. No. 18/133,991 is also a Continuation In Part of U.S. patent application Ser. No. 17/211,721, filed Mar. 24, 2021, and entitled “VAPORIZERS WITH CARTRIDGES WITH OPEN SIDED CHAMBER” which is a Continuation In Part of U.S. Pat. No. 10,986,872 filed Aug. 30, 2018 entitled “VAPORIZER AND VAPORIZER CARTRIDGES” which is a Continuation of U.S. Pat. No. 10,076,137, filed Feb. 17, 2016 entitled “VAPORIZER AND VAPORIZER CARTRIDGES” which claims the benefit of U.S. Provisional Pat. Applications Ser. No. 62/116,926 entitled CARTRIDGE AND HEATER filed on 17 Feb. 2015; Application Ser. No. 62/127,817 entitled MULTI ZONE VAPORIZER filed on 3 Mar. 2015; Application Ser. No. 62/184,396 entitled VAPORIZER DEVICE AND METHOD 25 Jun. 2015; Application Ser. No. 62/208,786 entitled VAPORIZER CARTRIDGE AND HEATER 23 Aug. 2015; Application Ser. No. 62/270,557 entitled THIN CONVECTION VAPORIZER filed 21 Dec. 2015 the disclosures of each of the above referenced applications and patents are incorporated by reference herein in their entirety as if fully set forth herein.

This application is a Continuation of U.S. patent application Ser. No. 18/412,194 entitled “VAPORIZING CONSUMABLES HEATED WITH CONVECTION AND CONDUCTION IN A PORTABLE DEVICE” filed Jan. 12, 2024 which is a Continuation in Part of U.S. patent application Ser. No. 18/133,991 (also U.S. Pat. No. 11,979,949) filed Apr. 12, 2023 entitled “PORTABLE MULTIZONE INDUCTION VAPORIZER FOR TOBACCO CONSUMABLES” the disclosures of each of the above referenced applications and patents are incorporated by reference herein in their entirety as if fully set forth herein.

This application is a Continuation of U.S. patent application Ser. No. 18/621,058 entitled “PORTABLE NON-COMBUSTION VAPORIZER FOR TOBACCO CONSUMABLES” filed Mar. 28, 2024, which is a Continuation In Part of U.S. patent application Ser. No. 18/412,194 the disclosures of each of the above referenced applications and patents are incorporated by reference herein in their entirety as if fully set forth herein.

This application is a Continuation In Part of U.S. patent application Ser. No. 18/233,240 entitled CONTROLLED HEATING OF PLANT MATERIAL IN DISPOSABLE CARTRIDGES WITHOUT COMBUSTION″ filed Aug. 11, 2023 which claimed the benefit of U.S. Provisional Pat. Application Ser. No. 63/437,698 entitled “VAPORIZATION CARTRIDGE ZONED HEATING” filed Jan. 8, 2023 and is also a Continuation In Part of U.S. Pat. No. 11,707,877 filed Jan. 12, 2021 the disclosures of each of the above referenced applications and patents are incorporated by reference herein in their entirety as if fully set forth herein.

### BACKGROUND OF THE DISCLOSURE

### Field of the Disclosure

The present disclosure relates generally to non-combustible disposable cartridges which can heat without burning or combusting vaporizable oils, compounds including those from plant-based material such as tobacco, hemp and cannabis. A consumable with frangible portions is configured to insert into a heating chamber wherein the plant-based material is heated by one or more of conduction and heated air via convection without combustion.

### Related Art

Cannabis, hemp, tobacco and other botanicals have been known in the art to be vaporized or burned to release organic material in the form of inhalable material. Vaporizing at correct temperatures can boil off the oils for inhalation without combusting the plant material.

Vaporization allows aromatherapy or inhalation. Herbs and botanicals have been known in the art to be vaporized or burned to release organic material in the form of inhalable material.

Lavender vaporizes at 260° F. Tobacco vaporizes between 257° F. to over 392° F. Green tea vaporizes between about 175° C. to over 185° C. Valerian vaporizes at about 235° C. Chamomile used to aid in the relief of anxiety vaporizes at about 380° F. Peppermint vaporizes at about 255° F. Peppermint is also known to ease symptoms of allergies and asthma, in addition to alleviating some of the side effects that come along with the common cold or a sinus infection. Cannabis, has a range at which it can be heated to release different cannabinoids as vapor without burning the organic material from below 200 F to about 430 F.

Heating a cartridge configured to contain organic plant material and/or infused oils on a carrier material may, in some instances, overheat at least portions thereof and therefore combust, overheat or otherwise release unwanted substance which may include carcinogens and chemicals into the vapor.

Uneven heating of a consumable in a heating chamber can result in uneven use of the consumable, charring and in some instances combustion.

It is therefore a desideratum to have a device, method and or system wherein such heating avoids combustion and/or uneven heating.

### DESCRIPTION

A method, system and device is disclosed which can heat not burn plant material such as tobacco in a consumable cartridge through a fluid pathway including a consumable cartridge with an inhalation end, containment end containing one or more of tobacco, hemp and cannabis and one or more spacers extending at least a portion of the exterior diameter of said consumable; and, wherein the extended exterior diameter is sufficient to orient the consumable within a receivor whereby at least a portion of the consumable containment is separated from the inner wall of the receivor.

A method, system and device is disclosed which can heat not burn plant material such as tobacco in a consumable cartridge through a fluid pathway including a consumable cartridge with an inhalation end, containment end containing one or more of tobacco, hemp and cannabis and one or more spacers extending at least a portion of the exterior diameter of said consumable; and, wherein the extended exterior diameter is sufficient to orient the consumable within a receivor whereby at least a portion of the consumable containment is separated from the inner wall of the receivor and wherein the spacer encircles less than the entire circumference of the consumable; and a slot where the spacer is not encircling the consumable forms an airflow pathway. In some instances the slot is at least one serpentine pathway.

A method, system and device is disclosed which can heat not burn plant material such as tobacco in a consumable cartridge through a fluid pathway including a consumable cartridge with an inhalation end, containment end containing one or more of tobacco, hemp and cannabis and one or more spacers extending at least a portion of the exterior diameter of said consumable and wherein the extended exterior diameter is sufficient to orient the consumable within a receivor whereby at least a portion of the consumable containment is separated from the inner wall of the receivor. The spacer extends an uneven height from the consumable towards the receivor inner wall and the lower height portions form airflow pathway.

In some instances the spacer is thermally conductive; and, a region of material including at least one of a flavor filter, flavoring, flavor within tobacco compounds and other flavor within non-tobacco compounds is positioned inside consumable in thermal communication with the conductive spacer. In some instances the one or more spacers encircles at least 40% of the consumable. In some instances at least two airflow pathways are formed. In some instances the one or more spacers encircles at least one of 50%, 60%, 70%, 80%, 90% and 95% of the consumable. In some instances a spacer is positioned adjacent to the containment end of the consumable. In some instances a spacer is positioned adjacent to the inhalation end of the consumable.
1. A method, system and device is disclosed which can heat not burn plant material such as tobacco in a consumable cartridge through a fluid pathway and said consumable is configured to deform upon insertion into a duct or receivor. Said consumable forms a tube which may be cylindrical or polyhedron and has an inhalation end, containment end containing one or more of tobacco, hemp and cannabis. wherein one or more portions of the consumable are frangible and configured to bend inward when passing over a protrusion within or adjacent to a receivor and when inserted into a receivor a and one or more spacers extending at least a portion of the exterior diameter of said consumable; and, wherein the extended exterior diameter is sufficient to orient the consumable within a receivor whereby at least a portion of the consumable containment is separated from the inner annular wall of the receivor. In some instances the spacer encircles less than the entire circumference of the consumable; and, a slot where the spacer is not extending the diameter of the consumable forms one of a space for air between the consumable and the inner wall of the receivor and an airflow pathway. In some instances a divider located at a distal end of the material and a filter located within the inhalation end.

### DRAWINGS

The invention may be better understood by referring to the following figures. The components in the figures are not necessarily to scale, emphasis instead being placed upon illustrating the principles of the invention. In the figures, like reference numerals designate corresponding parts throughout the different views.

FIGS. 1-4D illustrate aspects of cartridge modules and associated heater.

FIGS. 5A-5B illustrate aspects of cartridge modules and associated heater with multi- zone heating configuration.

FIGS. 6A-6H illustrate aspects of a cartridge heating system with heating chamber key portion.

FIGS. 7A-7C illustrate aspects of a disposable cartridge and heating chamber.

FIGS. 8 illustrates aspects of a cartridge and key portion heating chamber.

FIGS. 9A illustrates aspects of a disposable cartridge and key portion heating chamber.

FIGS. 9B to 9E illustrate aspects of a disposable cartridge and key portion chamber of FIG. 9A along the line of “A” to “A”.

FIG. 9F illustrates a partial view of FIG. 9B showing aspects of a disposable cartridge and key portion heating chamber.

FIGS. 10A and 10E illustrate aspects of a protrusion for one or more of deforming a frangible section of a consumable and limiting input of the consumable in receivor.

FIGS. 10F and 10J illustrate aspects of a protrusion for limiting input in the receivor and/or a spacer for positioning part of the consumable remote from the receivor inner wall and forming an air passage/air gap.

FIGS. 11A to 11E illustrates aspects of a Key Hole heating chamber for a non-combustible consumable cartridge heating system.

FIGS. 12 to 14 illustrates processes of control of heating for heating a removable non-combustible consumable cartridge.

All descriptions and callouts in the Figures and all content therein are hereby incorporated by this reference as if fully set forth herein.

### FURTHER DESCRIPTION

Combustion free heating for a disposable consumable cartridge which is an elongated tube formed of one or more materials which wrap at least a material to vaporize therein. The material to vaporize includes tobacco (or other plant material) to release smoke free vapors which is consider healthier for consumer than combustion and burning of substances such as tobacco. Disclosed herein are aspect of the use of a disposable consumable cartridge having one or more deformable frangible sections and said consumable cartridge wrapping may contain metal foil regions. Said consumable with material therein is insertable into a controlled heating device. During or before insertion of the consumable into a receivor, an actuator deforms the frangible section(s) of the consumable. In some instance the deformation forms an air gap in a separation between at least the frangible deformed section of the consumable and the receivor inner wall. A controller receiving temperature sensor(s) input controls the supply of power to heating elements thereby heating the consumable and air in the gap.

It is appreciated by those skilled in the art that some of the circuits, components, controllers, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs*), time-to-frequency conversations, frequency-to-time conversions, database mapping, signal processing steps, coding, modulations, demodulations, etc. The controller devices and smart devices disclosed herein operate with memory and processors whereby code is executed during processes to transform data, the computing devices run on a processor (such as, for example, controller or other processor that is not shown) which may include a central processing unit (“CPU”), digital signal processor (“DSP”), application specific integrated circuit (“ASIC”), field programmable gate array (“FPGA”), microprocessor, etc. Alternatively, portions DCA devices may also be or include hardware devices such as logic circuitry, a CPU, a DSP, ASIC, FPGA, etc. and may include hardware and software capable of receiving and sending information.

Heating logic turns on/off heating elements forming zones to heat different sections of the cartridge at different times. In some instances the cartridge has limited orientations of insertion to hold it fixed in the heater and unable to rotate about its axis. In some instances the cartridge is marked with a frangible identifier which is broken on insertion to prevent reuse of a spent cartridge. In some instances the cartridge is marked with an identifier that is stored in memory to turn off the heater if the cartridge has already been used.

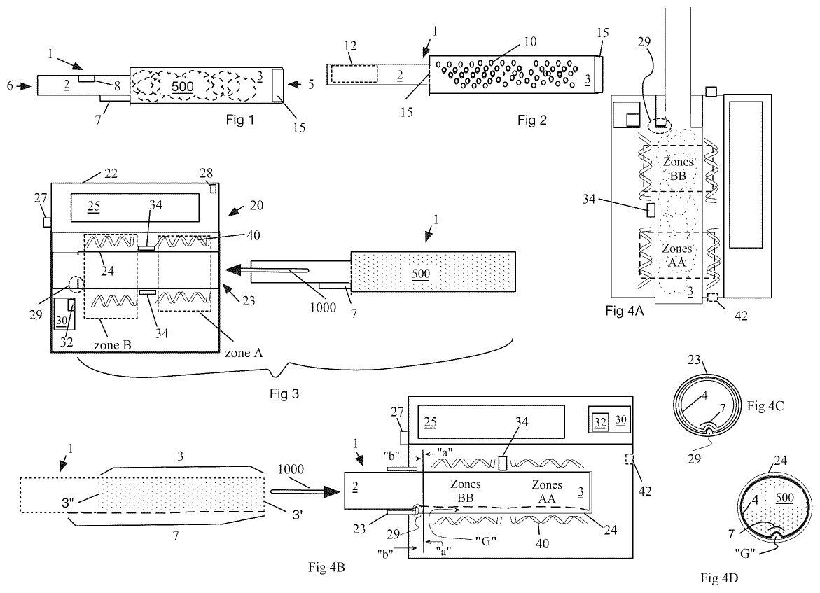

FIGS. 1, 2, 3 and 4A and 4B show cartridge 1 with two ends, the first end 2 is an inhalation (or intake) end or portion and the second end 3 is a containment (or heating) end or portion. The cartridge is generally tubular and holds material to be heated. During use fluid (air) passes into the open front 5 to the distal boundary 3′ of the containment end 3, through the material 500 past the proximal boundary 3″ of the containment end cartridge and then into the inhalation end 2 and finally out through the open back 6. Optionally, a frangible section 7 may be formed on the cartridge whereby it will be deformed during use with a heater. In some instances the deformation may render the cartridge finished an unable to be reused. In some instance an ID 8 which verifies cartridges non-used status may be added to the cartridge. In some instances small perforations 10 may be formed in the containment end 3 to effectuate better heat flow from heating elements. In some instances a filter or flavor filter 12 is placed within the inhalation end 2 whereby vapor inhaled passes. The filter can remove some materials from the vapor and the flavor filter adds an inhalable flavor to the vapor. A flow through divider 15 such as a spacer, filter, screen or coarse filter which allows fluid to pass through may be positioned within the outer wrap 4 of the cartridge. Organic matter 500 is placed within the containment 3 for use of the cartridge. The organic material is a material containing oils or resins (such as, hemp, tobacco and cannabis) which can be released via heating.

The cartridge is formed of an inexpensive disposable material which will not burn or release toxic or harmful fumes at temperatures that are reached by the heater in the device. The outer wrap 4 encases the within components of the consumable cartridge. In generally for many organic materials the temperature of vaporization will be between 320 F to 450 F. The cartridge may be scarred by the heating process as it is disposable. Paper, fibers such as cotton and hemp, metal, foil, plastic, resins, thermoplastics, ceramics, ceramic doped paper, glass, PEEK, and combination thereof may be suitable material for some or all of the cartridge. The cartridge maybe made of different materials for different regions. For example the containment portion 3 is subjected to the greatest heat. The material or materials therein must be suitable to transfer a sufficient portion of the heat applied to its surface through its wall and into the containment portion to thereby cause vapor of the organic material 500 without burning. In some instances the interior annular wall of the containment portion has one or more conductive regions facing the inside.

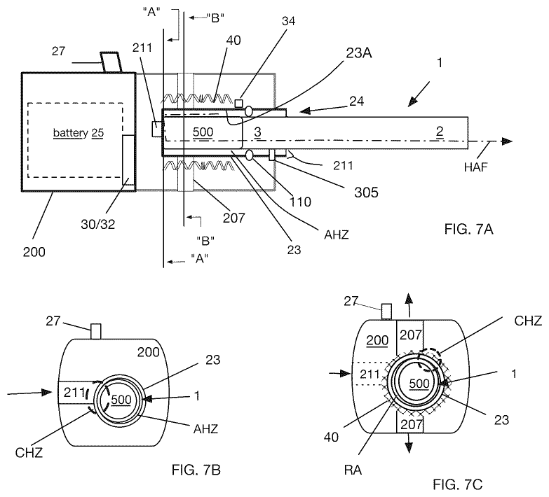

FIGS. 3 and 4D illustrate aspects of a consumable heating with combustion device. During use the cartridge 1 inserted into a heater 20 via the pathway of arrow 1000. This also may be referred to as a pass-through cartridge device as the cartridge guide is open at both end. The example of the passing the cartridge into the heater is not a limitation and those of ordinary skill in the art will recognize that a non-pass-through configuration is within the scope of this disclosure which is also described in reference to FIGS. 7A, 8-11C. The heater 20 has a case 22 with an interface fluidly connected to a receivor 24 (also referred to as a cartridge guide). The interface 23 opens into a cartridge guide 24. The cartridge guide (receivor) is a channel within the case that is open to allow passage of the cartridge therein. The guide refers to a region within the case that is roughly the cross-sectional circumference or perimeter around the cartridge. Accordingly, if heating elements are placed in the area of the cartridge guide, they would be adjacent to at least a portion of the cartridge. Within the case is a battery 25. A cartridge identification reader 26 may also be added to the case. The identification reader is a sensor that verifies a cartridge is new and has not been previously used. It may determine that a heat indicator has previously been heated, it may determine that a code which is ablated by the heat of use his or is not present thereby interrupting or allowing heating. It may read a code and verify that the code has not been used during a prescribed interval. An on/off switch 27 is shown, and battery may have a charging I/O 28. The case may have a mechanical or electrical mechanical actuator 29 which protrudes in the insertion path of a cartridge and deforms a cartridges frangible section 7 upon insertion. The actuator positioned in or near one of the interface 23 and a receivor 24 (also referred to as a cartridge guide) which are fluidly connected. The deformation of said frangible section may actuate a sensor. Actuation is a communication of the actuator 29 to the controller whereby the controller recognizes the state of the cartridge as “new” and not used and thereby allows electrical current to flow to the heating elements. Within the case is a controller 30. The controller is a microprocessor which may have memory 32 and which controls certain operations of the vaporizer device. Operations may include one or more of time, date, location, security code, on/off, sequence of heating, temperature, indicator display of the heater, battery charging, battery management, battery state of charge indication, communication via illumination, sound, vibration and the like with the user and cartridge verification. Those of ordinary skill in the art will recognize that blue tooth or other wireless or wired connection to a smart phone or computer may also be used to perform some of the controller functions and that would be within the scope of this disclosure. One or more temperature sensors 34 are within the case and near the receivor 24.

The case 22 contains one or more heating elements 40. One or more heater vents 42 may be provided. Both heating elements are shown those of ordinary skill in the art will understand that what is disclosed is one or more zones. In some instance only a single heating zone may be provided, in other instances multiple zones may be utilized and such is within the scope of this disclosure.

In some exemplary implementations a multi-zone heater is disclosed it may have heat zone “A” and “B”. A cartridge, during use, will have corresponding zones “AA”” to “BB” which align generally with the heat zones.

During use one or more zones may be turned on to supply heat, via heating elements, to heat organic material 500 and release vapor. Sequencing the zones for heating is advantageous in that it can reduce power consumption by splitting up the total area to heat into the zones so that less power per zone is needed then power to heat all zones at the same time. Sequencing the zones for heating is also advantageous in that it can release vapor from a discreet amount of organic material at one time thereby leaving less heated or unheated areas of organic material with the same cartridge for a next use. Sequential heating also reduces overheating and supports continuous use while reducing overheating. Overheating, for at least cannabis results in singeing the material which is commonly referred to as a “popcorn” taste.

FIGS. 4B to 4D illustrates aspects of a non-pass-through embodiment of the cartridge heating without combustion device shown in FIG. 4A. During use the cartridge 1 is inserted into a heater 20 via the pathway of arrow 1000. The heater 20 has a case 22 with an interface fluidly connected to a receivor 24 (also referred to as a cartridge guide) open at one end and in fluid communication with the interface 23. The receivor acts as a heating chamber. The cartridge guide (receivor) accepts insertion of the cartridge therein. The cartridge guide refers to a region within the case that is roughly the cross-sectional circumference or perimeter around the cartridge. Accordingly, if heating elements are placed in the area of the cartridge guide, they would be adjacent to at least a portion of the cartridge. Within the case is a protruding mechanical actuator associated with one of the interface and the cartridge guide. The actuator deforms a cartridge's frangible section 7 upon insertion through an interface 23 into a receivor 24 (also referred to as a cartridge guide), the deformation(s) form gaps “G” of air between portions of the cartridge guide and the cartridge. Within the case is a controller 30. The controller is a microprocessor which may have memory 32 and which controls certain operations of the vaporizer device. Operations may include one or more of time, date, location, security code, on/off, sequence of heating, temperature, indicator display of the heater, battery charging, battery management, battery state of charge indication, communication via illumination, sound, vibration and the like with the user. Those of ordinary skill in the art will recognize that blue tooth or other wireless or wired connection to a smart phone or computer may also be used to perform some of the controller functions and that would be within the scope of this disclosure. One or more temperature sensors 34 are within the case and near the receivor 24. FIG. 4C is a cross section along line “a”-“a” of FIG. 4B, a frangible section of the cartridge is deformed via passing over the protruding actuator into the cartridge guide 24. FIG. 4D is a cross section along line “b”-“b” of FIG. 4B, the frangible section of the cartridge is separated from the cartridge guide inner wall to having an indented area in cross section forming an air gap “G” between the receivor wall and indented frangible section. At least one of cartridge outer wrap 4 and the material is configured to indent (deform) when engaging the protrusion thereby forming a co-axial region with a smaller cross sectional diameter than the cartridge before it engages the protrusion.

During use one or more zones may be turned on to supply heat, via heating elements, to heat organic material 500 and release vapor. Areas of the cartridge in contact with the inner wall of the cartridge guide will heat by direct heat transfer from the wall to the cartridge, areas of air near the deformed frangible sections will heat up and be drawn through the cartridge upon inhalation. Sequencing the zones for heating is advantageous in that it can reduce power consumption by splitting up the total area to heat into the zones so that less power per zone is needed then power to heat all zones at the same time. Sequencing the zones for heating is also advantageous in that it can release vapor from a discreet amount of organic material at one time thereby leaving less heated or unheated areas of organic material with the same cartridge for a next use. Sequential heating also reduces overheating and supports continuous use while reducing overheating. Overheating, for at least cannabis results in singeing the material which is commonly referred to as a “popcorn” taste.

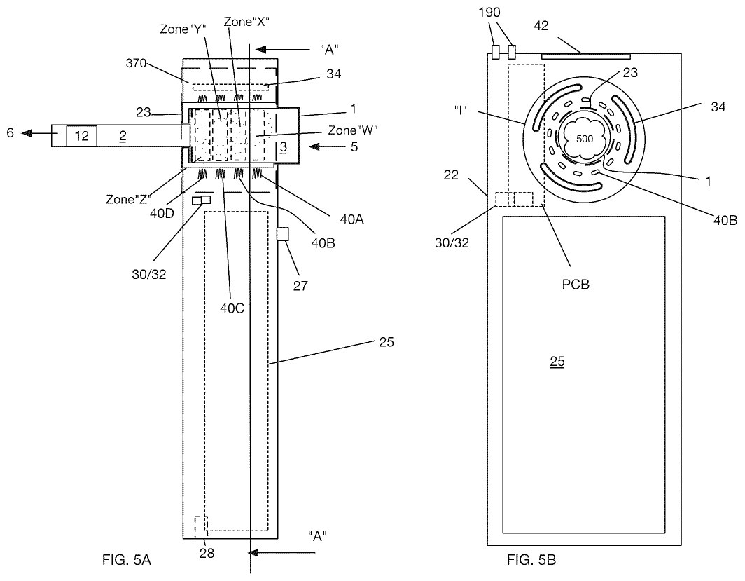

FIGS. 5A-5B show an alternative package for the systems shown in FIGS. 1-4. This disclosure heats the cartridge 1 akin to slicing a sausage. Each of zones “W”-“Z” are sequentially heated. The controller keeps count of which was the last zone to be heated. The controller keeps count of when all zones have been heated and can stop the heat cycle until a spent cartridge is removed and replaced with a new cartridge. It also adds indicators 190. Indicators are shown as LED lights. Indicators are illumination used to communicate status of the device to a user. The communication may be of a spent cartridge, a spent zone that has been heated, the remaining zones to heat, a need for recharge, or remaining zones to heat. Heating elements 40A-4D are in thermal contact with heat zones “W”-“Z”. One or more temperature sensors 34 are within the case and near the receivor 23, each temperature sensor is associate with at least one of a heat zone and heating element. Insulation “370” may be placed around the heater elements inside the case 22. The heater elements may optionally be connected to a PCB board via conductive wires and the controller and memory may also be on that board. The battery 25 is connected via the on/off switch 27 to the controller 30 to supply power to the heat elements. The controller 30 may be connected to separate digital memory 32. The controller initiates each heater element (40A-40D) sequentially to spend each zone and then use the next. When the sequence is complete the device stops heating until reset. All aspects of systems to verify, authenticate and assure that a used cartridge is not reloaded into the device, as described herein with respect to other exemplars are hereby incorporated into this description with respect to FIGS. 5A and 5B.

The cartridge mates with the receivor 23 which places it adjacent to heating elements. The cartridge heating portion (containment) should be constructed so that it does not burn, or combust at exposure temperatures below at least one of 400 degrees F., 410 degrees F., 420 degrees F., 430 degrees F., and 440 degrees F. The failure to burn or combust occurring after at least one of 30 seconds exposure, 1 minute exposure, 2 minute exposure. The failure to burn or combust occurring after at three least 30 second exposures. The failure to burn or combust occurring after at three least 1 minute exposures. The failure to burn or combust occurring after at least four 1 minute's exposures. The failure to burn or combust occurring after at five least 1 minute exposures. The failure to burn or combust occurring after at seven least 1 minute exposures. The failure to burn or combust occurring after at eight least 1 minute exposures.

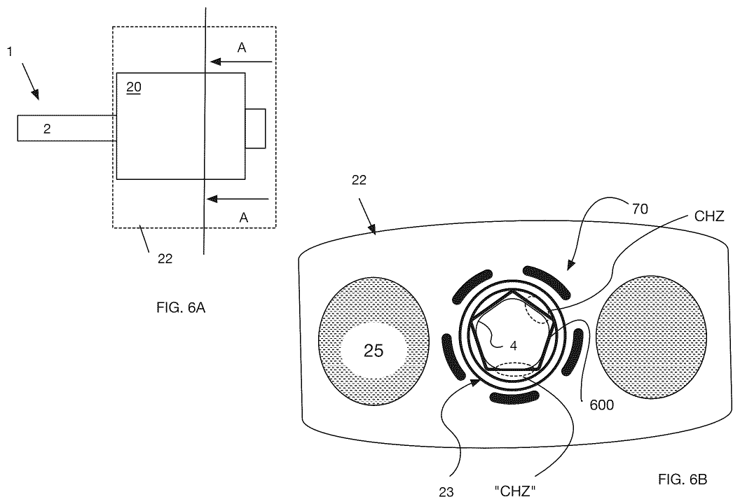

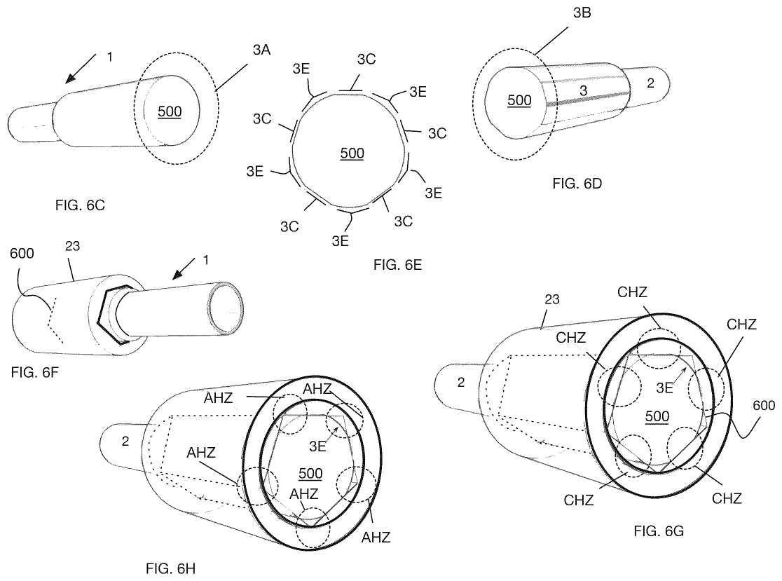

FIGS. 6A-6G shows aspects of another exemplary implementation of the cartridge and heater device. A heater 20 in a case 22 heats a disposable cartridge 1. FIG. 6B is a cut-away view from line “A-A” of FIG. 6A, it shows a receivor 23 and a key portion 600. One or more heating elements are shown. Said heating elements may be a series of vertical heating elements 70 (also referred to as an array). Said vertical heating elements are each aligned with a specific region of the heating chamber in an axial direction. In some instances the heater 20 is one or more heating elements encircle at least a part of the receivor (heating chamber) a previously described. The controller supplies power to each one or more heater element in response to temperature sensor data. In some instances the heating may be sequential the turning on/off of a heating element is controlled by the controller. The controller can also receive temperature sensor data to turn one or more heating elements or to maintain a set exposure temperature. The controller can turn on one of the heating elements thereby directing the heat to one heating zone. Memory either volatile or non-volatile will store data on system parameters when the controller is not powered. The controller instructs the on/off of heating elements within the heating array.

The disposable elongated cylindrical cartridges should be thin walled to effect heat transfer and malleable whereby its shape can be altered cross sectionally be pressure applied to it such as passing it over a key. The key portion 600 reshapes sections of the cartridge by forming indentations in the cylindrical cartridge. The key portion limits cartridge rotation about its axis when in the receivor. Limiting rotation of the consumable cartridge around its axis in the receivor aligns or places one or more predefined sections of the containment end second shaped end against regions of the receivor inner wall in predetermined locations. In some instances vertical heating elements can be aligned with the controller keeps count of which was the last zone to be heated.

Those of ordinary skill in the art will recognize that the illustration of the key portion as a pentagon shape is not a limitation and that any non-circular portion in the receivor (heating chamber) including but not limited to polygonal shapes and complex curves with indents and protrusions as part of the inner wall of the receivor (heating chamber). Conductive Heat Zones “CHZ” are regions wherein the shaped section(s) of the cartridge containment end are placed in predefined locations in direct physical contact with the receivor interior wall 23A. Conductive Heat Zones (“CHZ”) are configured for conduction of heat from one or more heaters through the wall of the receivor to the one or more sections of the cartridge in physical contact with the interior receivor wall. Viewed axially, at least the cartridge containment within the receivor (heating chamber) will have section in physical contact with the receivor and sections separated from the receivor wall by air gaps.

FIGS. 6C-6E shows a disposable consumable cartridge before and after insertion into the heater heating chamber (receivor) 23. Before insertion the containment end 3 has a circular first shape 3A. After insertion the containment end has a noncircular second shape 3B. FIG. 6E shows a cross section of the containment end after insertion into the heating chamber and key portion 600. As the key portion illustrated is pentagonal, after insertion of the containment end through the heating chamber key portion five flattened (or indented) sections 3C of the cartridge containment end are seen. Material 500 in the containment end will be compressed by the shaping.

FIGS. 6F, 6G and 6H show front and back perspective views of the cartridge in the receivor and the key. CHZs are called out where the cartridge contacts with the inner annular wall 23A. Other non-indented or unflatten sections 3E of the containment end are spaced apart from (not in physical contact with) the receivor's inner annular wall 23A forming Air Heating Zones “AHZ”. During use, the air in the AHZs is heated. Passing the cylindrical cartridge into the key portion of the receivor reshapes the cartridge section that passes therethrough. During use the sections of the shaped containment end 3B which are in physical contact with the inner annular wall are conduction heated and sections remote from the inner wall form volumes of air in the AHZ. In some instance vertical heater positioning corresponds to the key portions which are configured to indent the cartridge containment end to form CHZs. In some instance vertical heater positioning corresponds to the key portions which are configured to form AHZs. In some instance vertical heater positioning corresponds to the key portions which are configured to form one or more CHZs and one or more AHZs. The key portion prevents cartridge rotation about its axis when in the receivor (heating chamber). Limiting rotation of the consumable cartridge around its axis in the receivor aligns or places one or more predefined sections of the containment end second shaped end against regions of the receivor inner wall in predetermined locations. The controller counts and/or controls heating of each vertical heater such that each vertical heater is controlled. In some instance the heater(s) corresponding to one or more CHZ are activated, in other instance the vertical heater(s) corresponding to one or more AHZ are activated. In other instances a combination of vertical heaters corresponding to CHZ and AHZ are activated. In other instances selection of heating element materials to generate more heat in a particular area and/or arraying a group of heating filaments to have a denser region of filaments to provide more heat per unit area near a predetermined area is disclosed. In other instances, using dissimilar heating filaments with the ones corresponding to the predetermined area being less conductive and/or providing less heat per unit area. The heating element is configured to providing different heat per unit area around the receivor.

FIGS. 7A-7C show a non-pass through cartridge heating system, the cartridge distal end is kept in the receivor by the receivor. A case 200 contains the one or more heating elements 40. Heating elements include a coil wire, Kapton™ (polyamide) or silicone tape with metalized flat elements, iron-chromium-aluminum (FeCrAl) alloys, nichrome (nickel chrome alloy) wires, filaments or any material which does not outgas at the desired temperatures, are located around the outside annular wall of the receivor 23 (heating chamber). The cartridge fits into the case 200 via a cartridge interface 24 which is, part of and/or fluidly connected to the receivor 23. Said receivor is both a guide for accepting a cartridge and heating chamber. Upon insertion of the containment end 3 (which is also referred to as distal end) of the cartridge into the heating chamber (also referred to as a receivor) 23. Said receivor is configured to be in thermal communication with heat element(s) surrounding at least part of the receivor. Said thermal communication refers to the transfer of heat provided by one or more heating elements through the heating chamber wall. The cartridge 1 is removable from the case. The inhalation end 2 (also referred to as the proximal end) is extended from the case. The receivor 23 may have seals 110 to form a better closure between receivor and cartridge. An organic plant material 500 for vaporization is within the distal end near the one or more heating elements 40. A positional sensor 305 can provide input to the controller to decision if a cartridge is in the receivor. Said controller uses such data to control the timing and amount of power provided to the one or more heating elements.

During use, a volume of air in the receivor in an Air Heating Zone “AHZ” between the inner wall 23A and at least the cartridge is heated when heating element(s) heat the inner wall 23A of the receivor and the cartridge inside the receivor. The volume of heated air will flow during inhalation on the inhalation end forming a convection air flow also referred to as a Heated Airflow “HAF” which is drawn from the distal end of the consumable cartridge distal end (containment) 3 to and through the proximal end 2. One or more vents 207 allow the heater to vent from the case. At least one air intake 211 provides a fluid pathway for additional air to enter the case and be drawn through the cartridge from distal end to proximal end and then out for inhalation. The sections of the cartridge containment end in physical contact with the inner wall 23A are Conductive Heat Zones “CHZ” and also heat the material therein by conduction. FIGS. 7B shows a cut away view of the device along the line of A-A and FIG. 7C shows a cut-away of the device along the lines of “B-B”.

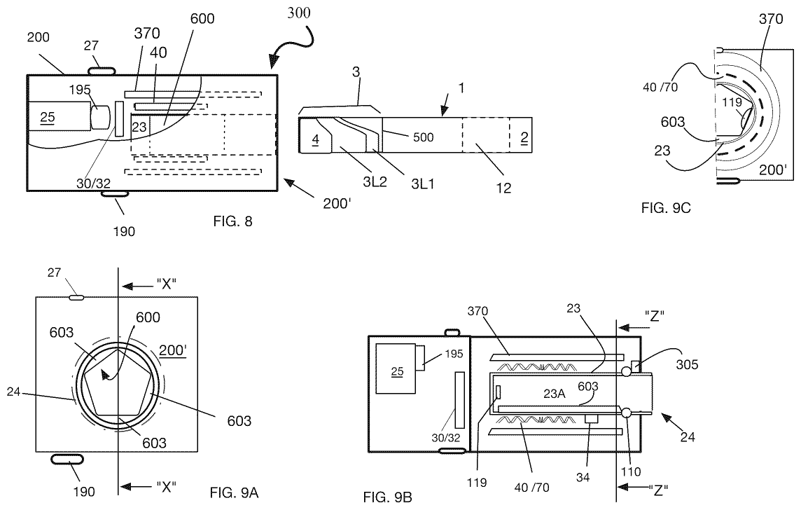

FIG. 8 shows a component overview of a consumable and heating system 300 with a key portion and heating chamber or receivor. The case 200 described above includes but is not limited to heating element(s), controller, temperature sensor(s) 34, power supply, illumination (communication) PCB, switches, and memory and all electronics placing the heating element(s) under the control of the controller which is configured to produce measured heat to a defined level. Said defined level may be fixed or variable. A communication display such as illumination via an electroluminescent screen, light emitting diode (LED) indicators 190 or a liquid crystal display may be added. It is also within the scope of the disclosure that communication with user may be via sound, or a vibration controlled by the controller. Said vibration producing means 195 include but are not limited to a DC motor or piezoelectric devices.

The cartridge 1 is an elongated cylindrical tube with a proximal end 2 for inhalation, which also may have a filter or added flavor 12 therein and a containment end 3 which contains plant material to be vaporized. The cartridge is formed of an inexpensive disposable material which will not burn or release toxic or harmful fumes at temperatures that are reached in the device. The cartridge may be scarred by the heating process as it is disposable. Paper, fibers such as cotton and hemp, metal, foil, plastic, resins, thermoplastics, ceramics, ceramic doped paper, glass, PEEK, and combination thereof may be suitable material for some or all of the cartridge. The cartridge maybe made of different materials for different regions. For example the containment portion 3 is subjected to the greatest heat. The material must be suitable to transfer a sufficient portion of the heat applied to its surface through its wall and into the containment portion to thereby cause vapor release from the organic material 500. In some instances the containment end has more than one layer including at least a conductive layer 3L1 containing metal, foil or other material which facilitates heat transfer and an outer layer 3L2. The outer wrap 4 is shown as the outermost layer, those of ordinary skill in the art will recognize that in some instance an additional layer which may be conductive may be placed over the outer wrap 4.

In use, the consumable cartridge's containment end 3 is placed into the open first end of a receivor 23. A key portion 600 of the heating chamber (receivor) extends into the receivor causing it to be at least partially non-circular. The key portion physically presses and/or compresses the cartridge tube indenting or shaping a portion of the cartridge and containment end to roughly follow the contour of the shape of the key portion.

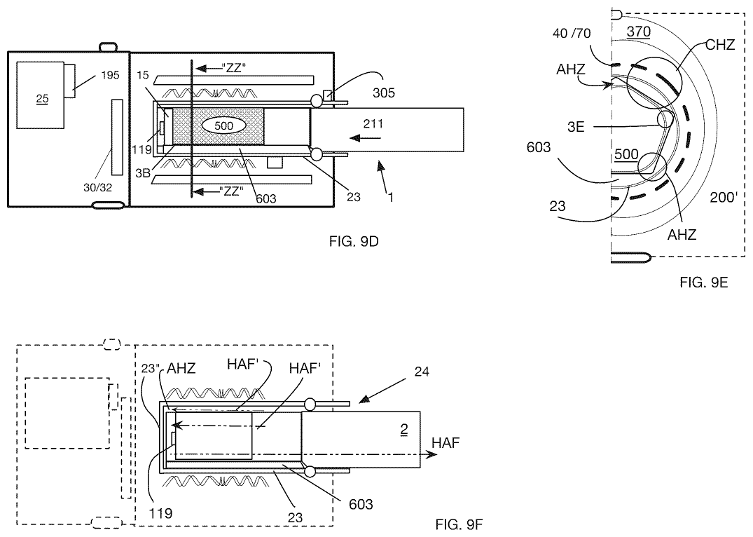

FIGS. 9A-9F show aspects of the heating system generally described herein utilizing a key portion 600 for cartridge positioning and/or creation of heating zones. FIG. 9A shows a front view of a portable heating system. At the front of the case 200′ is the cartridge interface 24 which is fluidly connected to the receivor whereby the cartridge is inserted into the heating chamber 23 and key portion for heating disposable consumables.

FIGS. 9B is a view along the line of “X”-“X” of FIG. 9A. The key portion 600 extends from the receivor forming non circular regions or protrusions 603, heating elements may be one or more heating element encircling at least part of the heating chamber 40 or vertical heating elements 70. FIG. 9C is a cut away view of FIG. 9B along the line “Z”-“Z” showing the key portion receivor. FIG. 9D is another cutaway of FIG. 9A along the line of “X”-“X” but has the disposable consumable cartridge 1 inserted. FIG. 9E is a cut away view of FIG. 9D along the line of “ZZ”-“ZZ” showing the compressing or indenting of the cartridge containment end via passage through the key portion indents sections of the cartridge containment end to fit tightly against the inner wall of the receivor forming Conductive Heat Zones “CHZ” and Air Heating Zones “AHZ”. The “CHZ” is a conduction region of heat transfer to the cartridge containment end and material therein when heating is active. The “AHZ” is adjacent to non-indented or unflatten sections 3E of the containment end and is heated by heat transfer through the receivor when heating is active whereby the volume of air in the “AHZ” is heated. In some instances one or more actuators 29 in the receivor are protrusion dividers 119′ formed in the receivor (heat chamber inner wall) said positioning divider can inhibit stops downward movement of an inserted consumable's distal end forming a gap of a pre-determined volume between the distal end and the receivor. In this fashion the material in the consumable is not compressed. When the consumable end is at the end 23″ of the non-pass through receivor some compression of the material may occur if the distal end of the consumable is forced against the end 23″ thereby limiting airflow through the material.

FIG. 9F is a partial view of FIG. 9B illustrating Heated Airflow “HAF” moving within the system. The volume of air heated in AHZ forms at least a portion of the Heated Airflow “HAF”. Such Heated Airflow “HAF” moves through the cartridge from containment end to inhalation end along the “HAF” arrow. Vapor absent combustion byproducts is released from the material 500 is carried with the air flow during use (inhalation). The heating at the CHZ also heats the material as part of the vapor, without combustion, process. In some instance vertical heaters 70 are positioned corresponds to the key portions which are configured to indent the cartridge containment end to form CHZ. In some instance vertical heaters are positioned corresponding to the key portions which are configured to form AHZ. In some instance vertical heater positioning corresponds to the key portions which are configured to form one or more CHZs and one or more AHZ. The key portion prevents cartridge rotation about its axis when in the receivor (heating chamber). Limiting rotation of the consumable cartridge around its axis in the receivor aligns or places one or more predefined sections of the containment end second shaped end against regions of the receivor inner wall in predetermined locations. The controller counts and/or controls heating of each vertical heater such that each vertical heater is controlled. In some instance the heater(s) corresponding to one or more CHZ are activated, in other instance the vertical heater(s) corresponding to one or more AHZ are activated. In yet other instances a combination of vertical heaters corresponding to CHZ and AHZ are activated.

Those of ordinary skill in the art will understand that any cartridge with an indented or shaped containment end whereby a portion of the containment end is in direct contact with the inner wall of the receivor and another portion is remote from the inner wall of the receivor is within the scope of this disclosure.

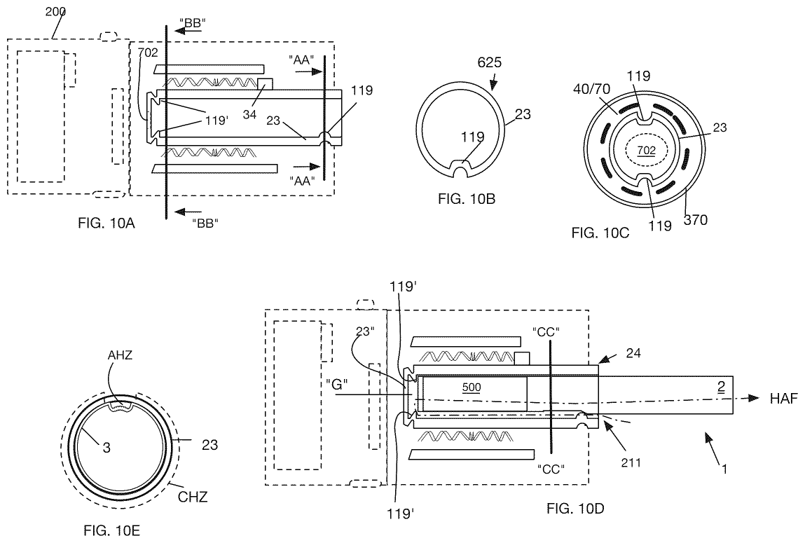

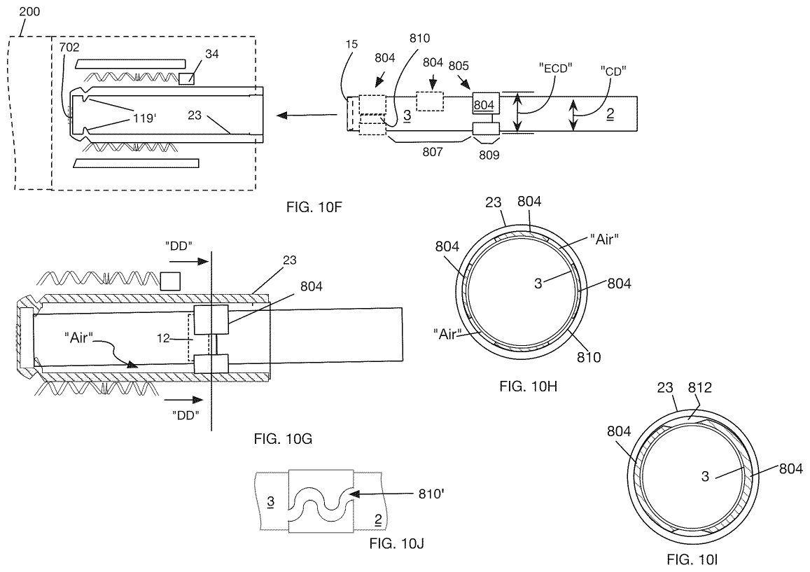

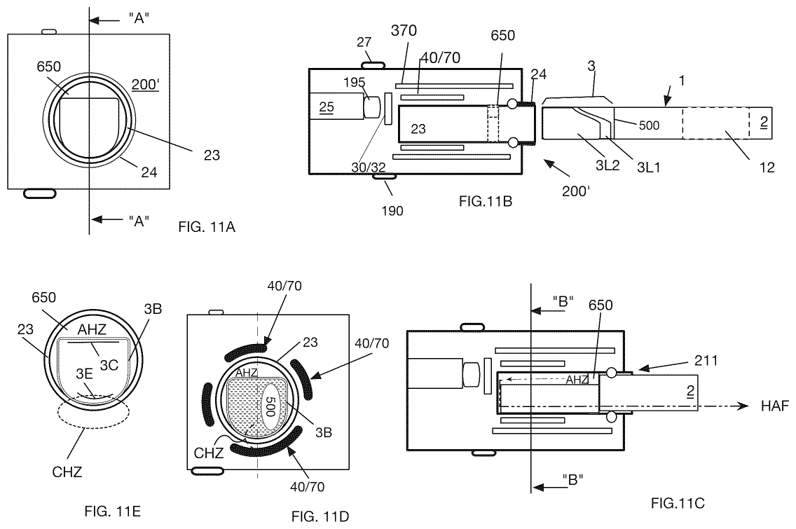

FIGS. 10A through 10E illustrate another Key Hole heating system and another heating chamber Key Hole 625 which compresses at least a portion of the containment end of a consumable cartridge when inserted into in a heating chamber whereby portions of the containment end are in physical contact with the heating chamber and portions are separated with an air gap in-between the containment end and the heating chamber. One or more Conductive Heat Zones “CHZ” and Air Heating Zones “AHZ” are formed corresponding to the areas of physical contact and the areas where air in the heating chamber (receivor) is heated as described in reference to the aspects of the exemplary implementation(s) described FIGS. 9A to 10E above. Extending inward from the heating chamber 23 thermal (heat) transfer inner wall of the heating chamber (receivor) is a protrusion 119 or protrusion divider 119′ which extend inward towards the center of the receivor. The receivor provides thermal communication from heating elements to the interior of the receivor or heating chamber. The protrusion may also have exchange properties and is constructed of a material and in a configuration to facilitate heat transfer. Upon insertion of the containment end of the consumable through the Key Hole 625, the protrusion 119 interferes with the passage of the consumable inward and one or more frangible sections of the consumable deform or indent forming one or more elongated Air Heating Zone(s) “AHZ” along the length of the consumable wherein air will be heated, other portions of the consumable containment end are in physical contact with the heating chamber and form the Conductive Heat Zone(s) “CHZ”. At the end 23″ of the receivor (heating chamber) the non-pass through system optionally one or more vents may be formed 702.

FIG. 10B is a cutaway along the line “AA”-“AA” of FIG. 10A in which the Key Hole 625 is shown. FIG. 10C is cutaway along the line “BB”-“BB” of FIG. 10A in which the protrusion dividers 119′ are shown. The one or more protrusions dividers extend sufficiently to inhibit or stops downward movement of an inserted consumable's distal end forming a gap “G” (see FIG. 10D) of a pre-determined volume between the distal end and the receivor 23″. In this fashion the material in the consumable is not compressed. When the consumable end is at the end 23″ of the non-pass through receivor some compression of the material may occur if the distal end of the consumable is forced against the end 23″ airflow through the material may be limited.

FIG. 10D illustrates a cut-away view of aspects of the heating system for consumables showing the Heated Airflow “HAF” through the consumable and the positioning of the consumable via the dividers 119. The protrusion dividers 119′ limit movement of the distal end of the consumable in the receivor thereby reducing compression at the material on the end of the consumable. In other instances a coarse filter 15 may be added which at the distal boundary of the containment end which also, or instead of, the protrusion dividers moves the material away from the distal end of the consumable thereby reducing compression. FIG. 10E is a cut-away view at line “CC”-“CC” of FIG. 10D illustrating Air Heating Zone “AHZ” and Conductive Heat Zone “CHZ” of a consumable within the receivor.

FIG. 10F-10J illustrate an alternate embodiment of the exemplar shown in FIG. 10A. A positioning spacer 804 is an extended portion of the consumable cartridge or a layer of said cartridge which encircles at least a portion of the consumable cartridge whereby the extended consumable diameter “ECD” of the consumable cartridge is greater than the consumable diameter “CD” via the spacer”. A leading edge 805 of the spacer 804 is configured to slide into the receivor. In some instances the spacer is configured to fit snuggly within a receivor thereby positioning said consumable and forming a separation between the consumable cartridge regions unaltered by spacer(s) 807 encirclement and the inner wall of the receivor 23. Air resides in the separation formed. The encircled portion of the consumable 809 is positioned closer to the receivor wall or against the receivor wall. In other instances the spacer has slots 810 or reduced height portions 812 which form air passageways. When the spacer has an uneven area 812 (FIG. 10I) the spacer is configured to orient at least a portion of the consumable cartridge regions unalerted by spacer(s) 807 encirclement separated from the inner wall of the receivor 23 forming a region of air passage “Air. The spacer is formed as part of, added as a layer or attached to the removable fluid path of the consumable thereby making portions of the exterior diameter of the consumable larger in diameter. In some instances the spacer is more conducive to heat than the unaltered portions 807 of the consumable cartridge, is configured to transfer heat from the wall of the receivor to the consumable cartridge. In some instances a flavor and/or a flavor filter 12 including compounds or other additive which may include tobacco or may be non-tobacco is placed in close to a thermally communication with conductive spacer whereby the spacer via the higher thermal conductivity directs heat to the region containing the flavor/flavor filter 12 or additive whereby during use the heat transfer will release vapor at the beginning of the use/vaporization sequence. Such earlier vaporization of the flavor or compound which may be in the material or adjacent to the material 500 improves the user experience of the heat without combustion consumable and system. Multiple spacers may be utilized. Placement close to the conductive spacer refers to a placement of at least some of the flavor material or filter closer to the conductive spacer than the placement of other material 500 without flavoring. The length of the consumable, location of the conductive spacer(s) and the heating profile of the receivor and/or the heated air therein may be coordinated to tailor the sequenced heating of flavoring as described within. Those of ordinary skill in the art will recognize that a spacer which encircles the consumable and has reduced height or uneven exterior portions 812 forms an air passage and a spacer which partially encircles the consumable and has more defined slots 810 between partial encirclements cach are within the scope of this disclosure. By way of the spacer a consumable cartridge will have multiple diameters. Where the outer diameter is less than the inner diameter of the receivor 23 are resides in the receivor and when the consumable is inserted into the receivor at least a portion of the consumable cartridge is spaced apart from the inner wall of the receivor forming the region where air is present. FIG. 10J illustrates a section of a consumable with a spacer configured with a serpentine 810′ slot or channel also known as an airflow pathway. The serpentine configuration increases the path length (versus a straight pathway) for the airflow. Configuring a selected length of airflow pathway and configuring the frequency of the serpentine path and the width of said serpentine pathway 810′ provides a flow control based on the cross section of the flow pathway over the length of said pathway. Accordingly, under a predetermined range of negative pressure applied the inhalation end of the consumable the flow rate of air through said slot may be set to a predetermined value thereby providing a setting which can be used to adjust timing of the application of heat (or the power supplied to a heating element) to control the heat without combustion for the material in the consumable.

Said air in the receivor is heated by the heating element(s) 40 as described previously regarding Air Heating Zones “AHZ”. In some instances the spacer may be oriented to one side of the consumable cartridge whereby a portion of the cartridge will be urged against the inner wall of the receivor forming a Conductive Heat Zones (“CHZ”) as described above. In some instances a filter or coarse filter 15 is placed at the distal boundary of the containment.

FIG. 11A-11E shows aspects of the use of a Key Hole 650 as an alternate shaping means for a cartridge which also forms Conductive Heat Zones (CHZ) and Air Heating Zones (HAF). A key hole 650 is configured to ident or shape the area of a consumable disposable cartridge passed therethrough.

FIG. 11A shows a front view of a portable heating system in a case 200′. A Key Hole 650 is visible inside the heating chamber (receivor) 23 which is fluidly connected to the cartridge interface 24 whereby the cartridge is inserted into the heating chamber 23 and Key Hole for heating disposable consumables cartridges. FIG. 11B shows the components with partial cut-away of the consumable and heating system.

FIG. 11C shows a cartridge 1 within the heating chamber after insertion through the Key Hole 650. The Key Hole is configured to ident sections of the cartridge inserted therethrough thereby both limiting the movement of the cartridge about its axis and positioning sections of the cartridge in predetermined regions of the heating chamber. An Air Heating Zone (AHZ) is shown wherein air heated via heat transferred from heating element(s) by heating chamber 23 travels through the cartridge as a Heated Airflow (HAF) carrying with it vapor released from the material within the containment end of the cartridge. FIG. 11D shows a cut away view from line “B”-“B” of FIG. 10C. It can be seen that sections of the second shape 3B of the containment end are heated conductively at CHZ and air volume in the AHZ is heated by the inner wall of the heating chamber.

FIG. 11E illustrates that in a Key Hole configuration the section of the cartridge's second shape 3B containment end is indented 3C and remoted from the inner wall and an AHZ with a volume of heatable air is formed between the indented area 3C of the second end and the inner wall. The section of the cartridge's second shape 3B containment end which is not indented 3E is in physical contact with the inner wall rather than being remote from that inner wall (as described in reference to a key portion 600) and it forms a CHZ which heats the containment portion via conduction. The result of the Key Hole passage is the formation of one or more areas of the cartridge is remote from physical contact with the inner wall and one or more areas of the cartridge is in direct physical contact with the inner wall.

FIGS. 10A through illustrate another Key Hole heating system and another heating chamber Key Hole 650A which compresses at least a portion of the containment end of a consumable cartridge in a heating chamber whereby portions of the containment end are in physical contact with the heating chamber and portions are separated with an air gap in-between the containment end and the heating chamber. One or more Conductive Heat Zones “CHZ” and Air Heating Zones “AHZ” are formed corresponding to the areas of physical contact and the areas where air in the heating chamber (receivor) is heated as described in reference to the aspects of the exemplary implementation(s) described FIGS. 9A to 10E above. Extending inward from the heating chamber 23 thermal (heat) transfer inner wall of the heating chamber (receivor) is a protrusion or divider 119 which provides thermal communication between the heating elements and the interior of the heating chamber. The protrusion has exchange properties and is constructed of a material and in a configuration to facilitate heat transfer. Upon insertion of the containment end of the consumable through the Key Hole 650A, the protrusion 119 indents a section of the consumable forming elongated an Air Heating Zone “AHZ” wherein air will be heated, other portions of the consumable containment end are in physical contact with the heating chamber and form the Conductive Heat Zone(s) “CHZ”. FIG. 11C is a cut-away view of FIG. 11B along arrows “C”-“C” showing the protrusion portion of the Key Hole 650A (See FIG. 11A), the Air Heating Zone “AHZ” in the second shape 3B containment end which is formed by passing the consumable through the Key Hole.

Methods disclosed include a controller that manages heating at a selected exposure temperatures (SET) to vaporize a portion of the material in the containment area in the accordance with one of variable, preselected and fixed times. The heating of all heating elements may also be referred to as a cycle or a heating cycle. When a cycle is over the cycle has timed out. Temperature sensors are utilized to measure when the chamber or subzone has reached a target temperature. If the amount of time a specific heating element is to be heated is reached the heating of that element has timed out. The controller can track, monitor, measure or otherwise count that heating time. In other instances the controller may switch between subzones, preferably using a PWM protocol to supply power to each heating element separately to maintain a temperature at a predetermined range. Selective heating allows the “off” zone to cool while the “on” zone is heating. Said cooling is effective to reduce singeing of cannabis material.

In some instances the controller prohibits heating when a zone has already been heated for a predetermined timeframe. In some instances the controller may accept a user over ride to allow reheating of a zone or to heat multiple zones simultaneously.

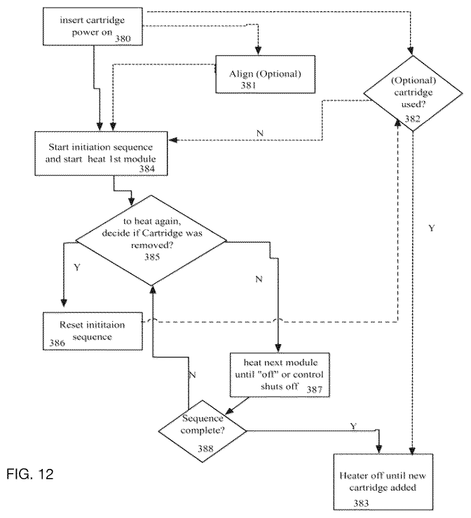

FIG. 12 illustrates aspects of a control sequence and sequence of operation of one or more exemplary implementations disclosed herein. First a cartridge is inserted into a heater unit and the on/off switch is depressed 380. Optionally, a mechanical, optical, or electro-mechanical fixture limits the orientation of the cartridge to a predefined one 381. Optionally, a sensor collects data on the cartridge to determine if it is used 382. If used 383 keep heater off until a new cartridge is added. If cartridge is not used then start power initiation and heat 1^{st} heating element/module 384 based on at least one of time and/or temperature, until user selects “off” or controller shuts off which may be due to time being completed. Then determine if cartridge has been removed 385. If the cartridge has been removed 386 then rest sequence of operation. If not removed 387 heat next heating element/module until user or controller shuts off. Next determine if sequence of heating is complete 388. The sequence may include heating one or more heating elements multiple times based on one or more of temperature measurements and time. If no heat cartridge if it has not been removed 385, else reset 386. If cartridge heating sequence is complete (all heating zones have been heated) then keep heater off until a new cartridge is provided 383.

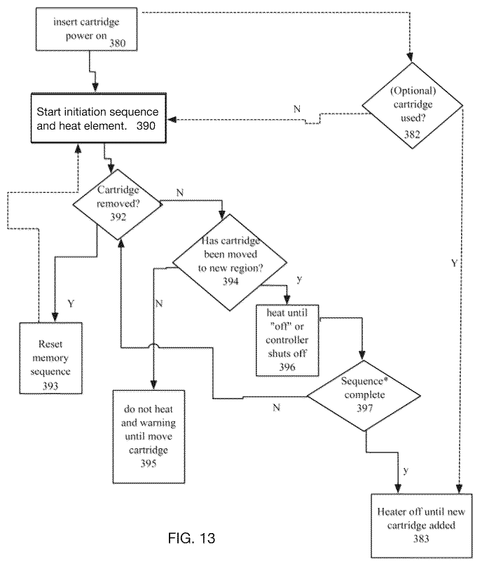

FIG. 13 illustrates aspects of a control sequence and sequence of operation of one or more exemplary implementations disclosed herein. First a cartridge is inserted into a heater unit and the on/off switch is depressed 380. Optionally, a sensor collects data on the cartridge to determine if it is used 382. Id used 383 keep heater off until anew cartridge is added. If cartridge is not used then start power initiation and heat heater element 390, heat until user selects “off” or controller shuts off. Determine if cartridge has been removed 392. If removed then rest sequence of operation 393. If not removed determine by position if cartridge has been rotated or slid to a new region 394. If not moved to new zone for heating, then do not heat and optionally warn with indicator, such a light, vibration or audible sound of need to move cartridge 395. If moved then heat until user or controller shuts off 396. Next determine if sequence of heating is complete 397. If no heat cartridge if it has not been removed 392 and if it has been moved 395.

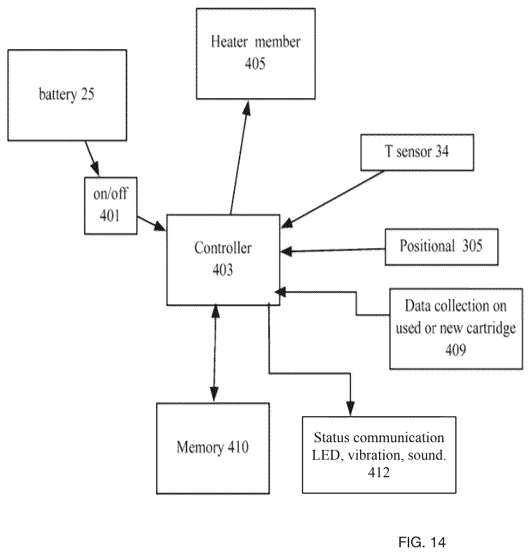

FIG. 14 shows aspects of an operational system for the systems and devices disclosed herein. A battery 25 is conductively connected to an on/off switch 401 then a controller 403. The controller 403 receives input from one or more of temperature sensor 34, positional sensor 305, data collection 409 (such as data on cartridge, RFID on cartridge, optical on cartridge) and communicates with Memory 410 optionally to determine status of cartridge—is it new or used? Has it been moved? Have all heating zones been heated sufficiently? The controller also can provide visual or audio feedback to the user via status indicators 412 such as LED lights, vibration or chirps like sounds from a microprocessor.

It will be understood that various aspects or details of the disclosures may be changed combined, or removed without departing from the scope of the invention. It is not exhaustive and does not limit the claimed inventions to the precise form disclosed. Furthermore, the foregoing description is for the purpose of illustration only, and not for the purpose of limitation. Modifications and variations are possible in light of the above description or may be acquired from practicing the invention. The claims and their equivalents define the scope of the invention.

What is claimed is:

1. A disposable consumable configured to spatially orient the consumable in a receivor comprising:
a cylindrical consumable cartridge with an inhalation end, containment end containing one or more of tobacco, hemp and cannabis and one or more spacers extending the cross sectional diameter of at least a portion of the exterior of said consumable; and,
wherein the extended exterior diameter is sufficient to orient the consumable within a receivor whereby at least a portion of the consumable containment is separated from the inner annular wall of the receivor.

2. The disposable consumable configured to spatially orient the consumable in a receivor of claim 1, wherein:
the spacer encircles less than the entire circumference of the consumable; and,
a slot where the spacer is not encircling the consumable forms an airflow pathway.

3. The disposable consumable configured to spatially orient the consumable in a receivor of claim 1, wherein the spacer extends an uneven height from the consumable towards the receivor inner wall and the lower height portions form airflow pathway.

4. The disposable consumable configured to spatially orient the consumable in a receivor of claim 1, wherein:
the spacer is thermally conductive; and,
a region of material including at least one of a flavor filter, flavoring, flavor within tobacco compounds and other flavor within non-tobacco compounds is positioned inside consumable in thermal communication with the conductive spacer.

5. The disposable consumable configured to spatially orient the consumable in a receivor of claim 2, wherein the one or more spacers encircles at least 40% of the consumable.

6. The disposable consumable configured to spatially orient the consumable in a receivor of claim 2, wherein at least two airflow pathways are formed.

7. The disposable consumable configured to spatially orient the consumable in a receivor of claim 2, wherein the one or more spacers encircles at least 50% of the consumable.

8. The disposable consumable configured to spatially orient the consumable in a receivor of claim 2, wherein the one or more spacers encircles at least 60% of the consumable.

9. The disposable consumable configured to spatially orient the consumable in a receivor of claim 2, wherein the one or more spacers encircles at least 70% of the consumable.

10. The disposable consumable configured to spatially orient the consumable in a receivor of claim 2, wherein the one or more spacers encircles at least 80% of the consumable.

11. The disposable consumable configured to spatially orient the consumable in a receivor of claim 2, wherein the one or more spacers encircles at least 90% of the consumable.

12. The disposable consumable configured to spatially orient the consumable in a receivor of claim 2, wherein a spacer is positioned adjacent to the containment end of the consumable.

13. The disposable consumable configured to spatially orient the consumable in a receivor of claim 2, wherein a spacer is positioned adjacent to the inhalation end of the consumable.

14. A disposable consumable configured to deform upon insertion into a duct or receivor comprising:
a cylindrical consumable cartridge with an inhalation end, containment end containing material which is at least one of tobacco, hemp and cannabis; and,
one or more portions of the consumable are frangible and configured to bend inward when passing over a protrusion within or adjacent to a receivor to form a space for a volume of air.

15. The disposable consumable configured to deform upon insertion into a duct or receivor of claim 14, wherein the one or more frangible portions remain bent inward after being affected by said protrusion.

16. The disposable consumable configured to deform upon insertion into a duct or receivor of claim 14, further comprising one or more spacers extending at least a portion of the exterior diameter of said consumable.

17. The disposable consumable configured to deform upon insertion into a duct or receivor of claim 16, wherein the extended exterior diameter is sufficient to orient the consumable within a receivor whereby at least a portion of the consumable containment end is separated from an inner annular wall of a receivor.

18. The disposable consumable configured to deform upon insertion into a duct or receivor of claim 16, wherein:
the spacer encircles less than the entire circumference of the consumable; and,
the partial encirclement is configured to form an airflow channel between the consumable and an inner wall of a receivor.

19. The disposable consumable configured to deform upon insertion into a duct or receivor of claim 18, wherein the channel is one of straight and serpentine.

20. The disposable consumable configured to deform upon insertion into a duct or receivor of claim 14, further comprising a divider located at a distal end of the material and a filter located within the inhalation end.
